(19) United States
(12) Patent Application Publication
(10) Pub. No.: US 2004/0131635 A1
(43) Pub. Date: Jul. 8, 2004

(54) Dehydrated vegetable product and method for obtaining same

(76) Inventors: Jean- Francois Colonat, Mollans Sur Ouveze (FR); Gilbert Ollivier, Aubignan (FR)

Correspondence Address: YOUNG & THOMPSON, 745 SOUTH 23RD STREET 2ND FLOOR, ARLINGTON, VA, 22202

(21) Appl. No.: 10/312,990

(22) PCT Filed: Jun. 26, 2001
(86) PCT No.: PCT/FR01/02016

(30) Foreign Application Priority Data
Jul. 3, 2000 (FR) 00/08574

(51) Int. Cl. A61K 35/80; A61K 35/78

(52) U.S. Cl. 424/195.17; 424/725

(57) ABSTRACT

The invention concerns a substantially dehydrated vegetable product, in particular a herb, an aromatic or spice plant, characterised in that it has the following characteristics: a mineral salt content of about 8 wt. % to about 15 wt. % relative to said vegetable product total weight; a carbohydrate or polyol content of about 4 wt. % to 15 wt. % relative to said vegetable product total weight; a water content of about 10 wt % to about 30 wt. % relative to the vegetable product total weight; a water activity (aw) of about 0.5 to about 0.7; preservation of organoleptic properties of the corresponding original product for a time interval of about 5 months.

The present invention relates to a dehydrated plant product, in particular of herb, aromatic plant, medicinal plant, spice type, and to a process for obtaining the said dehydrated plant product.

Freshly harvested plant products are for the most part available only at specific seasons and in a limited geographical area. Their storage time is short, particularly when they are cut so as to be ready for use. They are subject to microbiological degradations, to very rapid changes of flavours and colour, due principally to enzymes. They wither and their texture changes.

It is therefore particularly advantageous to dehydrate fresh plant products, in order to permit a lengthy preservation of their properties. Different dehydration techniques have been developed in order to have available plant products that are ready for use throughout the year, and the properties of which are preserved.

A first dehydration technique, which is the best known, is a traditional process which goes back to earliest times. It quite simply involves exposing cooked plant products to the sun. However, this process presents in particular the major drawback of the need for a large available surface for the implementation of the process.

A second, more modern, dehydration processes comprises lyophilisation. It involves a sublimation at low temperature carried out under vacuum to which certain products are subjected. A quick-freezing is carried out first, then the water is “sublimated”, creating a reduced pressure of 10⁻³ to 1 mm of Hg. During the quick-freezing, the water contained in the products changes from liquid into ice and, as a result, causes the cells of the product to burst, which thus allows the large molecules, and in particular those which produce or are flavours, to evaporate together with the water. This process therefore presents the drawback of causing the products to lose a large part of their organoleptic qualities.

A third process for the dehydration of plant products, which is also very well known, is drying with hot air. Hot air is circulated over the products to be dehydrated at a temperature of the order of 70° C. for tens of hours, which effectively amounts to a real cooking of the products, i.e. to a bursting of the cells, and consequently an even greater loss of their gustatory qualities.

Moreover, other documents have described treatments for the preservation of plant products by dehydration/osmotic impregnation.

In view of this, the subject of international application WO 92/10946 is a process for the preparation of dehydrated aromatic plants comprising a stage of immersing the plant to be dehydrated in a liquid bath, at a temperature of at least 82.2° C. (180° F.), containing an osmotic agent capable of infusing into the tissue of the plant and of forming an amorphous solid coating the plant during drying and a stage of drying the thus-infused and coated plant. An example of an osmotic agent is in particular a monosaccharide or a disaccharide. Thus according to the process described in this application, the sugar infuses the vegetable tissue of the plant and constitutes an amorphous solid which coats the surface of the dehydrated plant.

The principal drawback of coating is that it causes a crust to form on the treated products, which leads to a hard and brittle-texture. Moreover, during consumption, the saccharide which coats the plant produces a very sweet first sensation, although the dehydrated plants are used essentially in salty dishes.

At present, however, none of the processes of the prior art allows a dehydrated plant product to be obtained, the organoleptic properties of which are preserved for a period of several months, in particular at ambient temperature.

One of the aims of the present invention is to provide a dehydrated vegetable product in which the organoleptic properties of the corresponding original plant product are preserved for a period of several months, in particular at ambient temperature.

One of the other aims of the invention is to provide a particularly advantageous dehydration process for the obtaining of dehydrated products.

A subject of the present invention is a substantially dehydrated plant product, in particular a herb, aromatic plant or spice, characterised in that it presents the following characteristics:

a mineral salt content of approximately 8% to approximately 15% by weight relative to the total weight of the said plant product,

a glucide or polyol content of approximately 4% to approximately 15% by weight relative to the total weight of the said plant product, in particular approximately 5% to approximately 10%,

a water content of approximately 10% to approximately 30% by weight relative to the total weight of the said plant product, in particular approximately 13% to approximately 18%,

a water activity (wa) of approximately 0.5 to approximately 0.7, in particular approximately 0.55 to approximately 0.65,

the preservation of the organoleptic properties of the corresponding original plant product, for a period of at least approximately 5 months, and in particular approximately 5 months to approximately 12 months, at a temperature of at least approximately 15° C., in particular approximately 18° C. to approximately 22° C.

the plant product being such that it is substantially devoid of the starting flora present on the corresponding original plant product, that it is substantially devoid of the development of microorgansims, and that it presents an inactivation of the enzymes principally responsible for the degradation of the colour and smell.

By “substantially” dehydrated plant product is meant a product presenting a water content of approximately 10% to approximately 30% by weight relative to the total weight of the said plant product.

By “corresponding original plant product” is meant the fresh plant product, i.e. as freshly harvested, before any other transformation.

The organoleptic properties are principally the flavour, the taste, the texture and the visual appearance presented by the original plant product. By visual appearance is meant principally the colour but also anything which relates to the form and to the surface condition (smooth or granular, shiny or dull.)

The dehydrated plant product according to the invention is advantageously such that it presents an inactivation of the enzymes principally responsible for the degradation of the colour and of the smell of chlorophyll-containing plant products.

When the substantially dehydrated plant product presents a mineral salt content of less than 8%, the dehydration is insufficient and the obtained product keeps less well.

When the substantially dehydrated plant product presents a mineral salt content of more than 15%, the product is too dehydrated and presents a too-salty taste that is likely to mask the flavours.

A glucide or polyol content of less than 4% means an excessive salt impregnation.

A glucide or polyol content of more than 15% risks preventing salt impregnation and makes the product sticky, which can be a problem during subsequent drying.

A plant product in which the water content is less than 10% has a brittle texture and is rehydrated with difficulty when it is used.

A water content of more than 30% is harmful to preservation by making possible enzymatic actions and the development of microorganisms.

A plant product of which the water activity is less than 0.5 is too dry, its texture is not flexible and it is rehydrated poorly when it is used.

A water activity of more than 0.7 does not permit a good preservation at ambient temperature, because of microbiological risks (above all yeasts and moulds).

According to an advantageous embodiment of the invention, the plant product as 0.0 defined above also presents the preservation of the elasticity properties of the corresponding original plant product.

An elastic product resumes its starting shape after having been deformed or compressed, when the stress to which it has been subjected is cancelled (see example 4).

By plant product is meant in particular:

herbs and aromatic plants,

spices,

algae used for food purposes,

medicinal plants.

Herbs are in particular those chosen from the group constituted by dill, basil, parsley, chervil, coriander, cress, tarragon, mint, parsley or bay.

Aromatic plants are in particular those chosen from the group constituted by thyme, rosemary, savory, marjoram, oregano, sorrel or sage.

Aromatic plants also include flowers used for food flavouring purposes such as the rose, jasmin or orange flower.

Spices are in particular those chosen from the group constituted by ginger, green pepper, clove, pimento, juniper berries or pink peppers.

Medicinal plants are in particular those chosen from the group constituted by balm, vervain, camomile, passion flower, nettle, red vine, lavender or coltsfoot.

According to another advantageous embodiment of the invention, the plant product as defined above is characterised in that:

the mineral salt is chosen from the group constituted by halogenated salts and is in particular sodium chloride,

the glucide is chosen from the group constituted by the mono-, di- or oligosaccharides, and is in particular sucrose, fructose, glucose, lactose or maltose,

the polyol is in particular sorbitol or glycerol.

A subject of the present invention is also the use of a solution containing:

approximately 55% to approximately 70% of water, by weight relative to the total weight of the said solution, in particular approximately 65% to approximately 70%,

approximately 15% to approximately 30% of mineral salt, by weight relative to the total weight of the said solution, in particular approximately 20% to approximately 25%,

approximately 10% to approximately 30% of glucide or of polyol, by weight relative to the total weight of the said solution, in particular approximately 14% to approximately 25% of glucide or of polyol, and preferably approximately 14.5% to approximately 16.5% of polyol, and preferably approximately 19% to approximately 22% of glucide,

in order to obtain a substantially dehydrated plant product presenting a preservation of the organoleptic properties of the corresponding original plant product, for a period of at least approximately 5 months, and in particular approximately 5 months to approximately 12 months, at a temperature of at least approximately 15° C., in particular approximately 18° C. to approximately 22° C.

According to an advantageous embodiment of the invention, the use of the solution as defined above permits the carrying out by immersion at one and the same time of the blanching, the impregnation and the dehydration of a plant product, in particular of a herb, of an aromatic plant or of a spice.

By immersion is meant the act of dipping the plant in the aqueous solution containing the mineral salt and the glucide or the polyol. The result of the immersion is impregnation and partial dehydration.

By blanching is meant the operation of partial cooking applied to plants to ensure the inactivation of their enzymes. The plants are generally immersed in hot or boiling water.

A subject of the present invention is also a process for obtaining a substantially dehydrated plant product from a starting plant product, in particular a herb, aromatic plant or spice, at the end of which the organoleptic properties of the original plant product corresponding to the starting plant product are preserved long-term and at ambient temperature, the said process being characterised in that it comprises the following stages:

steeping of the starting plant product in a steeping solution containing:

approximately 55% to approximately 70% of water, by weight relative to the total weight of said solution, in particular approximately 65% to approximately 70%,

approximately 15% to approximately 30% of mineral salt, by weight relative to the total weight of the said solution, in particular approximately 20% to approximately 25%,

approximately 10% to approximately 30% of glucide or of polyol, by weight relative to the total weight of the said solution, in particular approximately 14% to approximately 25% of glucide or of polyol, and preferably approximately 14.5% to 16.5% of polyol, and preferably approximately 19% to approximately 22% for the glucide,

in order to obtain a partially dehydrated plant product,

drying of the partially dehydrated plant product obtained at the end of the steeping stage in order to obtain a substantially dehydrated plant product presenting:

a mineral salt content of approximately 8% to approximately 15% by weight relative to the total weight of the said plant product,

a glucide or polyol content of approximately 4% to approximately 15% by 0.0 weight relative to the total weight of the said plant product, in particular approximately 5% to approximately 10%,

a water content of approximately 10% to approximately 30% by weight relative to the total weight of the said plant product, in particular approximately 13% to approximately 18%,

a water activity of approximately 0.5 to approximately 0.7, in particular approximately 0.55 to approximately 0.65,

the preservation of the organoleptic properties of the original plant product corresponding to the starting plant product, for a period of at least approximately 5 months, in particular approximately 5 months to approximately 12 months, at a 0.0 temperature of at least 15° C., in particular approximately 18° C. to approximately 22° C.

The steeping stage carried out using the steeping solution as defined above advantageously permits the extraction of part of the water constituting the plant product, while transferring into the said plant product solutes from the steeping solution (impregnation).

Thus the steeping stage advantageously permits the blanching, the impregnation and the partial dehydration of the plant product to be carried out at one and the same time.

The starting plant product used in the process of the invention as described above can be:

a fresh plant product or a quick-frozen fresh plant product and,

a whole plant product or a part or parts of the said plant product such as stem(s), root(s), leaf (leaves) or flowers.

Moreover, the plant product used in the process of the invention as described above is in particular chosen from the group constituted by:

herbs and aromatic plants,

spices,

algae used for food purposes,

medicinal plants,

and in particular from: dill, basil, parsley, chervil, coriander, cress, tarragon, mint, parsley, bay, sorrel, oregano, thyme, rosemary, savory, marjoram, sage, ginger, green pepper, clove, pimento, juniper berries, pink peppers, balm, vervain, camomile, passion flower, nettle, red vine, coltsfoot, lavender, rose, jasmine flower or orange flower.

In the steeping solution used within the framework of the process of the invention as described above:

the mineral salt is chosen from the group constituted by the halogenated salts and is in particular sodium chloride,

the glucide is chosen from the group constituted by the mono-, di- or oligosaccharides, and is in particular sucrose, fructose, glucose, lactose or maltose,

the polyol is in particular sorbitol or glycerol.

According to an advantageous embodiment of the process of the invention, the steeping solution can also contain:

a compound containing the magnesium ion, such as that chosen from the group constituted by magnesium hydroxide, acetate, carbonate or chloride, in particular in a quantity of approximately 0.1% to approximately 0.3% by weight relative to the total weight of the steeping solution, and preferably approximately 0.2% and/or,

an acidifier such as citric acid, in particular in a quantity of approximately 0.1% to approximately 0.3% by weight relative to the total weight of the steeping solution.

The compound containing the magnesium ion permits the preservation of the whole of the chlorophyll-containing nucleus of the plant product, and will thus permit a substantially dehydrated plant product to be obtained the colour of which will be stable in the long term.

The role of the acidifier is double, as it serves first of all as a flavour enhancer then has a favourable microbiological effect, by lowering the pH.

According to another advantageous embodiment of the process of the invention, the steeping of the plant product in the steeping solution is carried out at a temperature of approximately 70° C. to approximately 120° C., in particular approximately 85° C. to approximately 100° C., for a period of approximately 2 minutes to approximately 15 minutes, in particular approximately 5 minutes to approximately 10 minutes.

According to another advantageous embodiment, the process of the invention comprises before the steeping stage:

a stage of washing the starting plant product, in particular in water optionally containing a food disinfectant such as sodium hypochlorite, the said washing stage being optionally followed by a stage of rinsing, in particular in water and,

a stage of spin-drying the plant product, in particular by centrifugation.

The centrifugation is carried out for example at a speed of approximately 300 revolutions per minute to approximately 900 revolutions per minute, for approximately 1 minute to approximately 3 minutes.

According to another advantageous embodiment, the process comprises at the end of the steeping stage a drying stage in order to obtain a partially dehydrated plant 0.5 product presenting:

a mineral salt content of approximately 5% to approximately 10% by weight relative to the total weight of the said plant product, in particular approximately 7% to approximately 9%,

a glucide or polyol content of approximately 2% to approximately 5% by 10 weight relative to the total weight of the said plant product, in particular approximately 3% to approximately 4%,

a water content of approximately 40% to approximately 60% by weight relative to the total weight of the said plant product,

a water activity of approximately 0.7 to approximately 0.9, in particular approximately 0.75 to approximately 0.85.

Thus, by “partially dehydrated plant product” is meant a plant product having a water content greater than that of the substantially dehydrated plant product obtained at the end of the different stages of the process of the invention as described above, but a water content less than that of the starting plant product.

Thus, in the above and in the following, the term partially dehydrated plant product describes the plant product obtained at the end of the steeping stage of the process of the invention and after spin-drying, containing 40% to approximately 60% of water.

According to an advantageous embodiment of the process of the invention, the drying of the plant product obtained at the end of the steeping stage is carried out by centrifugation, at a speed of approximately 300 revolutions per minute to approximately 900 revolutions per minute, for approximately 1 minute to approximately 3 minutes.

More particularly, the centrifugation is carried out firstly at a speed of approximately 300 revolutions per minute to approximately 400 revolutions per minute for approximately 1 minute then, secondly, at a speed of approximately 800 revolutions per minute for approximately 1 minute.

According to an advantageous embodiment of the process of the invention, the drying of the plant product obtained at the end of the steeping and spin-drying stage is carried out by blowing of hot air at controlled humidity, at a temperature of approximately 40° C. to approximately 70° C., for a period of approximately 5 minutes to approximately 30 minutes.

More particularly, the drying is carried out firstly at a temperature of approximately 55° C. to approximately 70° C. for approximately 5 minutes to approximately 15 minutes, then optionally secondly at a temperature of approximately 40° C. to approximately 45° C., for approximately 20 minutes to approximately 25 minutes. The drying is followed by a rest period the role of which is to permit the cooling down of the plant product and a rebalancing of the water content between the different parts of the plant. The rest period can vary from 3 to 48 hours and is preferably 5 to 9 hours.

According to another advantageous embodiment of the invention, the process comprises after the drying stage:

a stage of cutting the substantially dehydrated plant product, followed by a sorting stage to separate the leaves, the stems and the flowers of the aforementioned plant product,

a stage of packaging of the substantially dehydrated plant product, in particular under normal atmosphere or under vacuum, which can be kept either at the temperature of 18° C. to 22° C., or at 4° C., or at −20° C.

The aim of the cutting stage is to cut the substantially dehydrated plant product in order to obtain pieces measuring approximately 5 mm to approximately 7 mm. It can be carried out using a bi-dimensional cutter fed by a belt, producing cuts in the form of squares, strips or rods from the substantially dehydrated plant product. Numerous sizes of strips, rods or squares can be obtained by combining the different circular- or cross-cutting shafts of the cutter.

The sorting stage is in particular carried out by vibrating screening and on a densimetric column.

According to an advantageous embodiment, the process of the invention is characterised in that it comprises the following stages:

washing of the starting plant product, in particular in water optionally containing a food disinfectant such as sodium hypochlorite, the said washing stage being optionally followed by a stage of rinsing, in particular in water and,

spin-drying of the plant product, in particular by centrifugation, at a speed of approximately 300 revolutions per minute to approximately 500 revolutions per minute, for approximately 1 min to approximately 3 min,

steeping of the starting plant product in a steeping solution containing:

approximately 55% to approximately 70% of water, by weight relative to the total weight of the steeping solution, in particular approximately 65% to approximately 70%,

approximately 15% to approximately 30% of mineral salt, by weight relative to the total weight of the steeping solution, in particular sodium chloride, in particular approximately 20% to approximately 25%,

approximately 10% to approximately 30% of glucide, in particular chosen from the group constituted by sucrose, fructose, glucose, lactose or maltose or, approximately 10% to approximately 30% of polyol, in particular sorbitol or glycerol, by weight relative to the total weight of the steeping solution, in particular approximately 14% to approximately 25%, and preferably approximately 14.5% to 16.5% of polyol and preferably approximately 19% to approximately 22% of glucide, and optionally,

approximately 0.1% to approximately 0.3% of a compound containing the magnesium ion, such as magnesium hydroxide, acetate, carbonate or chloride, in particular 0.2% by weight relative to the total weight of the steeping solution,

the said steeping being carried out at a temperature of approximately 70° C. to approximately 120° C., in particular approximately 85° C. to approximately 100° C., for a period of approximately 2 minutes to approximately 15 minutes, in particular approximately 5 minutes to approximately 10 minutes,

spin-drying of the plant product obtained at the end of the steeping stage, in particular by centrifugation, at a speed of approximately 300 revolutions per minute to approximately 900 revolutions per minute, for approximately 1 minute to approximately 3 minutes, in order to obtain a partially dehydrated plant product,

drying of the partially dehydrated plant product obtained at the end of the spin-drying stage, in particular by blowing of hot air at controlled humidity, at a temperature of approximately 40° C. to approximately 70° C., for a period of approximately 5 minutes to approximately 30 minutes,

in order to obtain a substantially dehydrated plant product,

cutting of the substantially dehydrated plant product, followed by a sorting stage to separate the leaves, the stems and the flowers of the aforementioned plant product, which sorting is for example carried out by vibrating screening and on a densimetric column,

packaging of the substantially dehydrated plant product, in particular under normal atmosphere or under vacuum,

which can be kept either at the temperature of 18° C. to 22° C., or at 4° C., or at −20° C.

A subject of the invention is also a substantially dehydrated plant product capable of being obtained by implementation of the process as described above.

### DESCRIPTION OF THE FIGURES

FIG. 1 represents the characterisation of the texture of the parsley substantially dehydrated according to the process of the invention, under mechanical stress. More particularly FIG. 1 represents the resistance force and the elasticity (in Newtons) in relation to time (in seconds) of the substantially dehydrated parsley.

FIG. 2 represents the characterisation of the texture of the fresh parsley under mechanical stress. More particularly FIG. 2 represents the resistance force and the elasticity (in Newtons) as a function of time (in seconds) of the fresh parsley.

The following examples illustrate the invention, without limiting it in any way.

### EXAMPLE 1

### Preparation of Substantially Dehydrated Parsley

The starting plant product used in the example below is fresh parsley. The different stages of the process of the invention are described below:

washing of 10 kg of fresh parsley in clear water for 3 minutes, followed by a stage of rinsing in clear water for 1 minute,

first spin-drying of the parsley by centrifugation, at a speed of 400 revolutions per minute, for approximately 1 minute,

steeping of the parsley at a temperature of 85° C. for 6 minutes in a steeping solution containing:

64.3% of water,

20.9% of sodium chloride (NaCl),

14.6% of sorbitol,

0.2% of magnesium hydroxide (MgOH),

second spin-drying of the parsley by centrifugation, carried out firstly at a speed of 400 revolutions per minute for 1 minute, then secondly at a speed of 800 revolutions per minute for 1 minute,

in order to obtain 3.07 kg of partially dehydrated parsley containing:

42% of water,

7% of sodium chloride (NaCl),

4% of sorbitol,

a water activity of 0.810,

drying of the partially dehydrated parsley at a temperature of 60° C. for 14 minutes, by blowing of hot air at 20% relative humidity,

in order to obtain 2.14 kg of substantially dehydrated parsley containing:

16.7% of water,

10.5% of sodium chloride (NaCl),

5.7% of sorbitol,

a water activity of 0.635,

cutting of the substantially dehydrated parsley obtained in the stage described above, and retrieval of 2.07 kg of pieces of substantially dehydrated parsley,

sorting of the pieces of substantially dehydrated parsley, and retrieval of the leaves or pieces of leaves, the stems having been discarded (sorting yield=60%) in order to obtain 1.25 kg of substantially dehydrated parsley compared with 2.07 kg of parsley from the cutting.

Thus, from 10 kg of fresh parsley, 1.25 kg of substantially dehydrated parsley was obtained; the weight reduction factor is therefore 8 compared with the fresh parsley.

### CONCLUSION

The organoleptic qualities of the dehydrated product are very close to those of the fresh product and are maintained for more than 12 months at 20° C. and 24 months at 4° C. The essential oil content is a good indication of the aromatic quality of the product. The following values, expressed in mg/kg relative to the dry matter, were measured:

| | |
|---|---|
| fresh parsley | 6.550 |
| parsley substantially dehydrated according to the invention | 4.100 |
| parsley dehydrated by drying (comparative example) | 2.700 |

### EXAMPLE 2

### Preparation of Substantially Dehydrated Tarragon

The starting plant product used in the example below is fresh tarragon. The different stages of the process of the invention are described below:

washing of 10 kg of fresh tarragon in clear water for 3 minutes, followed by a stage of rinsing in clear water for 1 minute,

first spin-drying of the tarragon by centrifugation, at a speed of 400 revolutions per minute, for approximately 1 minute,

steeping of the tarragon at a temperature of 85° C. for 6 minutes in a steeping solution containing:

64.2% of water,

20.9% of sodium chloride (NaCl),

14.6% of sorbitol,

0.2% of magnesium hydroxide (MgOH),

0.1% of citric acid,

second spin-drying of the tarragon by centrifugation, carried out firstly at a speed of 400 revolutions per minute for 1 minute, then secondly at a speed of 800 revolutions per minute for 1 minute,

in order to obtain 4.70 kg of partially dehydrated tarragon containing:

41% of water,

7% of sodium chloride (NaCl),

3% of sorbitol,

a water activity of 0.821,

drying of the partially dehydrated tarragon at a temperature of 60° C. for 17 minutes, by blowing of hot air at 20% relative humidity,

in order to obtain 3.42 kg of substantially dehydrated tarragon containing:

18.9% of water,

9.6% of sodium chloride (NaCl),

4.1% of sorbitol,

a water activity of 0.668,

cutting of the substantially dehydrated tarragon obtained in the stage described above, and retrieval of 3.31 kg of pieces of substantially dehydrated tarragon,

sorting of the pieces of substantially dehydrated tarragon, and retrieval of the leaves or pieces of leaves, the stems having been discarded; the sorting yield is 50%;

1.65 kg of substantially dehydrated tarragon are obtained from 3.31 kg of tarragon before sorting.

Thus, from 10 kg of fresh tarragon, 1.65 kg of substantially dehydrated tarragon were obtained; the weight reduction factor is therefore 6 compared with the fresh tarragon.

### CONCLUSION

The organoleptic qualities of the dehydrated product are very close to those of the fresh product and are maintained for more than 12 months at 20° C. and 24 months at 4° C. The essential oil content is a good indication of the aromatic quality of the product. The following values, expressed in mg/kg relative to the dry matter, were measured:

| | |
|---|---|
| fresh tarragon | 17.000 |
| tarragon substantially dehydrated according to the invention | 16.000 |
| tarragon dehydrated by drying (comparative example) | 6.700 |

### EXAMPLE 3

### Preparation of Substantially Dehydrated Basil

The starting plant product used in the example below is fresh basil. The different stages of the process of the invention are described below:

washing of 10 kg of fresh basil in clear water for 3 minutes, followed by a stage of rinsing in clear water for 1 minute,

first spin-drying of the tarragon by centrifugation, at a speed of 400 revolutions per minute, for approximately 1 minute,

steeping of the tarragon at a temperature of 100° C. for 6 minutes in a steeping solution containing:

67.8% of water,

16% of sodium chloride (NaCl),

16% of sorbitol,

0.2% of magnesium hydroxide (MgOH),

second spin-drying of the basil by centrifugation, carried out firstly at a speed of 400 revolutions per minute for 1 minute, then secondly at a speed of 806 revolutions per minute for 1 minute,

in order to obtain 2.68 kg of partially dehydrated basil containing:

43% of water,

10% of sodium chloride (NaCl),

5% of sorbitol,

a water activity of 0.831,

drying of the partially dehydrated basil at a temperature of 60° C. for 15 minutes, by blowing of hot air at 20% relative humidity,

in order to obtain 1.85 kg of substantially dehydrated basil containing:

17.3% of water,

14.5% of sodium chloride (NaCl),

7.2% of sorbitol,

a water activity of 0.646,

cutting of the substantially dehydrated basil obtained in the stage described above, and retrieval of 1.79 kg of pieces of substantially dehydrated basil,

sorting of the pieces of substantially dehydrated basil, and retrieval of the pieces of leaves, the stems having been discarded; a yield (after sorting) of substantially dehydrated basil of 53% (0.95 kg of basil obtained from 1.79 kg of basil before sorting) is achieved.

Thus, from 10 kg of fresh basil, 0.95 kg of substantially dehydrated basil is obtained; the weight reduction factor is therefore 10.5 compared with the fresh basil.

### CONCLUSION

The organoleptic qualities of the dehydrated product are very close to those of the fresh product and are maintained for more than 12 months at 20° C. and 24 months at 4° C. The essential oil content is a good indication of the aromatic quality of the product. The following values, expressed in mg/kg relative to the dry matter, were measured:

| | |
|---|---|
| fresh basil | 5.000 |
| substantially dehydrated basil according to the invention | 2.500 |
| lyophilised basil (comparative example) | 2.000 |

### Example 4

### Assessment of the Texture of the Substantially Dehydrated Plant Product

The texture of the substantially dehydrated plant product compared with the texture of the fresh starting plant product is assessed by force measurement thus expressing its resistance with the help of a traction/compression machine.

The substantially dehydrated plant product studied below is parsley.

The material used within the framework of the determination of the texture of the substantially dehydrated plant product is listed below:

traction/compression machine of the ADAMEL-LHOMARGY brand and of DY31, type

10 Newtons force sensor,

plastic measurement probe: plastic cylinder 20 mm (diam.)×50 mm (height)

AUTOTRAC driving software,

measurement vessel: plastic pot with a diameter of at least 65 mm.

The measurement procedure is described below:

type of test: compression and driving in elongation,

temperature of the sample: ambient,

number of measurements: 3 measurements on 3 samples.

résumé of the test programme:

starting force: 0.2 N

penetration: −13 mm at 20 mm/min (stage 1)

stability at this position of −13 mm for 3 minutes (stage 2)

rapid rise of 2 mm i.e. a position of −11 mm at 200 mm/min (stage 3)

stability at this position of −11 mm for 2 minutes (stage 4),

parameters measured: average of the 3 tests of the force values (Newton) measured at stages 1, 2, 3 and 4.

Stage 1 represents the resistance force to the penetration of the probe. Stage 2 represents the resistance force of the plant exerted on the fixed probe. Stage 3 represents the elasticity upon withdrawal of the probe. Stage 4 represents the elasticity of the plant on the fixed probe.

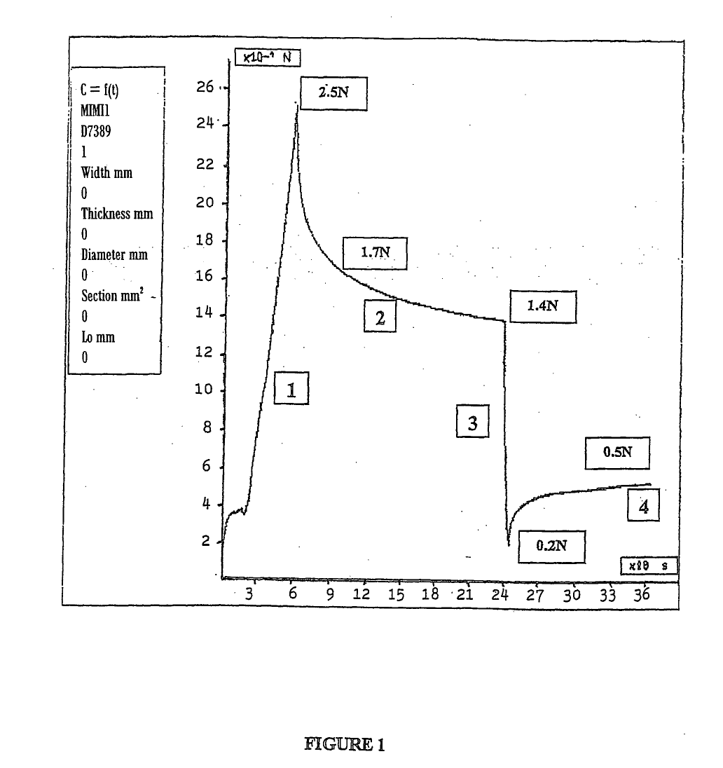

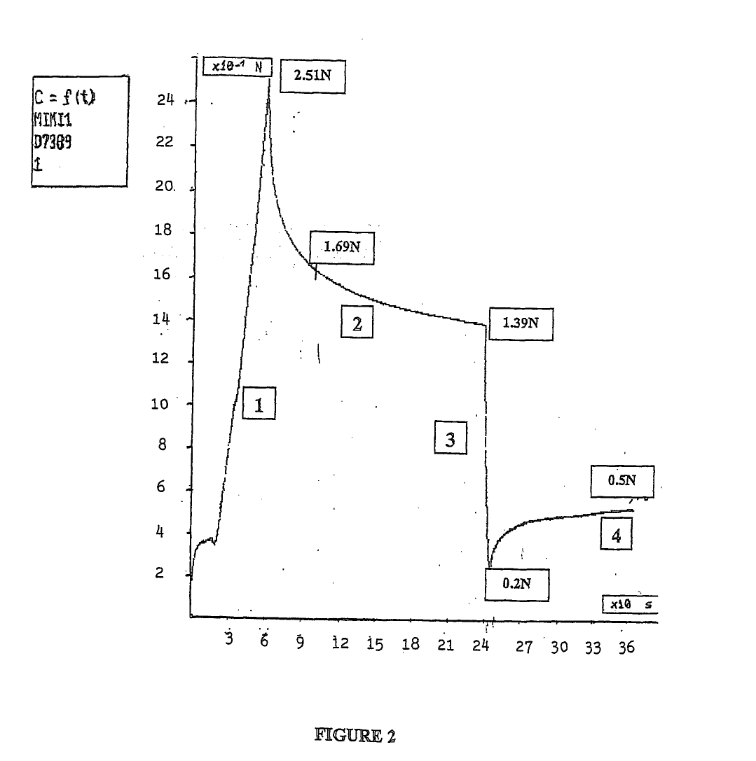

These 4 stages are represented in FIG. 1, which represents the resistance force and the elasticity (in Newtons) as a function of time (in seconds), of the substantially dehydrated parsley, and in FIG. 2 resulting from the test on the texture on fresh parsley.

Results:

Starting from these 4 force values and the curve of FIG. 1, the parameters characterising the texture of the substantially dehydrated plant product can be assessed compared with the texture of the fresh starting product. The comparaison between FIGS. 1 and 2 leads to the following conclusions:

the peak at the end of phase 1 is called “maximum force after compression”; this force, characteristic of the integrity of the structure of the product, is 2.51 N for the fresh parsley, as against 2.5 N for the substantially dehydrated parsley;

a “relaxation percentage” is defined by relating the force at the end of phase 2 to the maximum force; in this case, this gives 1.39/2.51 or 55.3% for the fresh parsley and 1.4/2.5 or 56% for the substantially dehydrated parsley;

the last parameter is the “elasticity percentage” which is the relationship between the force at the end of phase 4 and the maximum force; it is 0.53/2.51 or 21% for the fresh parsley and 0.5/2.5 or 20% for the substantially dehydrated parsley.

Comparing FIGS. 1 and 2, it will be seen that they are practically superimposable, which permits the affirmation that the texture of the substantially dehydrated parsley of the invention is virtually the same as that of the fresh starting parsley.

### EXAMPLE 5

Measurement of Enzymatic Activities

Common market garden parsley, freshly gathered, kept for 2 days at 4° C. is subjected to the dehydration process according to the invention, then the polyphenoloxydase activity and the peroxydase activity are measured.

The reaction mediums are the following:

| | |
|---|---|
| Polyphenoloxydase | Catechol 32 mM (final concentration) |
| activity: | Mc Ilvaine buffer, pH 6.8 - 0.2 M |
| | (final concentration) (at the temperature of the |
| | reaction (23° C.) the concentration of dissolved |
| | oxygen - second substrate for the enzymatic |
| | activity - is of the order of 0.25 mM) |
| | [spectrophotometric monitoring at 390 nm] |
| Peroxydase activity: | Gaiacol 25 mM (final concentration) |
| | Hydrogen peroxide 10 mM |
| | (final concentration) |
| | Mc Ilvaine buffer, pH 6.8 - 0.2 M |
| | (final concentration) |
| | [spectrophotometric monitoring at 470 nm] |

In both cases, the final volume of reaction medium was 3 ml, with 0.015 ml and 0.100 ml tests samples of enzymatic extracts.

All the activities tests took place at 23° C.

The extracts prepared from the samples of parsley, according to the invention, did not present significant polyphenoloxydase activity.

The extracts of parsley heated to 82° C., prepared according to the invention, have no significant peroxydase activity.

### Example 6

Comparative microbiological results between plants treated according to the process of the invention and fresh products.

Table I below gives the microbiological results obtained with parsley, tarragon, basil, fresh and treated according to the invention.

The procedure was carried out by spreading on nutrient mediums appropriate to each flora researched.

TABLE I

| | | | | Herbs treated | | |
|---|---|---|---|---|---|---|
| | | | | according | | |
| | Fresh herbs | | | to the | | |
| | Raw | | | invention | | |
| | Materials | | | Finished Products | | |
| | Parsley | Basil | Tarragon | Parsley | Basil | Tarragon |
| Total | 10⁶-10⁷ | 10⁶-10⁷ | 10⁴-10⁵ | <10³ | <10³ | <10³ |
| germs | | | | | | |
| Yeasts | 10⁴-10⁵ | 10⁴-10⁵ | 10³-10⁴ | <50 | <50 | <50 |
| Moulds | 10³-10⁴ | 10³-10⁴ | 10³-10⁴ | <50 | <50 | <50 |

### EXAMPLE 7

Comparative results for organoleptic quality between products treated according to the invention, fresh products, lyophilised products and products dehydrated according to the processes of the prior art.

The essential oil content is given in ppm per gram of dry product.

It is measured by gas chromatography. The results are given in table II below.

TABLE II
Essential Oils ppm/dry

| | Fresh | Invention | Lyophilised | Dehydrated |
|---|---|---|---|---|
| Parsley | 6550 | 4100 | 2730 | 1000 |
| Basil | 5000 | 2500 | 2000 | 2000 |
| Tarragon | 17000 | 16000 | 6700 | 6250 |

1. Substantially dehydrated plant product, in particular a herb, aromatic plant or spice, characterised in that it presents the following characteristics:
a mineral salt content of approximately 8% to approximately 15% by weight relative to the total weight of the said plant product,
a glucide or polyol content of approximately 4% to approximately 15% by weight relative to the total weight of the said plant product, in particular approximately 5% to approximately 10%,
a water content of approximately 10% to approximately 30% by weight relative to the total weight of the said plant product, in particular approximately 13% to approximately 18%,
a water activity (wa) of approximately 0.5 to approximately 0.7, in particular approximately 0.55 to approximately 0.65,
the preservation of the organoleptic properties of the corresponding original plant product, for a period of at least approximately 5 months, and in particular approximately 5 months to approximately 12 months, at a temperature of at least approximately 15° C., in particular approximately 18° C. to approximately 22° C.
the plant product being such as it is substantially devoid of the starting flora present on the corresponding original plant product, that it is devoid of the development of microorgansims and that it presents an inactivation of the enzymes principally responsible for the degradation of the colour and of the smell.

2. Plant product according to claim 1, characterised in that it presents the preservation of the elasticity properties of the corresponding original plant product.

3. Plant product according to claim 1 or claim 2, characterised in that it is in particular chosen from the group constituted by:
herbs and aromatic plants,
spices,
algae used for food purposes,
medicinal plants,
and in particular from: dill, basil, parsley, chervil, coriander, cress, tarragon, mint, parsley, bay, sorrel, oregano, thyme, rosemary, savory, marjoram, sage, ginger, green pepper, clove, pimento, juniper berries, the pink peppers, balm, vervain, camomile, passion flower, nettle, red vine, coltsfoot, lavender, rose, jasmine flower or orange flower.

4. Plant product according to one of claims 1 to 3, characterised in that:
the mineral salt is chosen from the group constituted by the halogenated salts and is in particular sodium chloride,
the glucide is chosen from the group constituted by the mono-, di- or oligosaccharides, and is in particular sucrose, fructose, glucose, lactose or maltose,
the polyol is in particular sorbitol or glycerol.

5. Use of a solution containing:
approximately 55% to approximately 70% of water, by weight relative to the total weight of the said solution, in particular approximately 65% to approximately 70%,
approximately 15% to approximately 30% of mineral salt, by weight relative to the total weight of the said solution, in particular approximately 20% to approximately 25%,
approximately 10% to approximately 30% of glucide or of polyol, by weight relative to the total weight of the said solution, in particular approximately 14% to approximately 25% of glucide or of polyol, and preferably approximately 14.5% to approximately 16.5% of polyol, and preferably approximately 19% to approximately 22% of glucide
in order to obtain a substantially dehydrated plant product presenting a preservation of the organoleptic properties of the corresponding original plant product, for a period of at least approximately 5 months, and in particular approximately 5 months to approximately 12 months, at a temperature of at least approximately 15° C., in particular approximately 18° C. to approximately 22° C.

6. Use of a solution according to claim 5, in order to carry out by immersion at one and the same time the blanching, the impregnation and the dehydration of a plant product, in particular of a herb, of an aromatic plant or of a spice.

7. Process for obtaining a substantially dehydrated plant product from a starting plant product, in particular a herb, aromatic plant or spice, at the end of which the organoleptic properties of the original plant product corresponding to the starting plant product are preserved long-term and at ambient temperature, characterised in that it comprises the following stages:
steeping of the starting plant product in a steeping solution containing:
approximately 55% to approximately 70% of water, by weight relative to the total weight of the said solution, in particular approximately 65% to approximately 70%,
approximately 15% to approximately 30% of mineral salt, by weight relative to the total weight of the said solution, in particular approximately 20% to approximately 25%,
approximately 10% to approximately 30% of glucide or of polyol, by weight relative to the total weight of the said solution, in particular approximately 14% to approximately 25% of glucide or of polyol, and preferably approximately 14.5% to 16.5% of polyol, and preferably approximately 19% to approximately 22% for the glucide,
in order to obtain a partially dehydrated plant product
drying of the partially dehydrated plant product obtained at the end of the steeping stage in order to obtain a substantially dehydrated plant product presenting:
a mineral salt content of approximately 8% to approximately 15% by weight relative to the total weight of the said plant product,
a glucide or polyol content of approximately 4% to approximately 15% by weight relative to the total weight of the said plant product, in particular approximately 5% to approximately 10%,
a water content of approximately 10% to approximately 30% by weight relative to the total weight of the said plant product, in particular approximately 13% to approximately 18%,
a water activity of approximately 0.5 to approximately 0.7, in particular approximately 0.55 to approximately 0.65,
the preservation of the organoleptic properties of the original plant product corresponding to the starting plant product, for a period of at least approximately 5 months, in particular approximately 5 months to approximately 12 months, at a temperature of at least 15° C., in particular approximately 1° C. to approximately 22° C.

8. Process according to claim 7, in which the starting plant product can be:
a fresh plant product or a quick-frozen fresh plant product and,
a whole plant product or a part or parts of the said plant product such as stem(s), root(s), leaf (leaves) or flowers.

9. Process according to Claim 7 or claim 8, in which the plant product is in particular chosen from the group constituted by:
herbs and aromatic plants,
spices,
algae used for food purposes,
medicinal plants,
and in particular from: dill, basil, parsley, chervil, coriander, cress, tarragon, mint, parsley, bay, sorrel, oregano, thyme, rosemary, savory, marjoram, sage, ginger, green pepper, clove, pimento, juniper berries, pink peppers, balm, vervain, camomile, passion flower, nettle, red vine, coltsfoot, lavender, the rose, the jasmine flower or orange flower.

10. Process according to one of claims 7 to 9, characterised in that in the steeping solution:
the mineral salt is chosen from the group constituted by the halogenated salts and is in particular sodium chloride,
the glucide is chosen from the group constituted by the mono-, di- or oligosaccharides, and is in particular sucrose, fructose, glucose, lactose or maltose,
the polyol is in particular sorbitol or glycerol.

11. Process according to one of claims 7 to 10, characterised in that the steeping solution can also contain:
a compound containing the magnesium ion, such as that chosen from the group constituted by magnesium hydroxyde, acetate, carbonate or chloride, in particular in a quantity of approximately 0.1% to approximately 0.3% by weight relative to the total weight of the steeping solution, and preferably approximately 0.2% and/or,
an acidifier such as citric acid, in particular in a quantity of approximately 0.1% to approximately 0.3% by weight relative to the total weight of the steeping solution.

12. Process according to one of claims 7 to 11, characterised in that the steeping of the plant product in the steeping solution is carried out at a temperature of approximately 70° C. to approximately 120° C., in particular approximately 85° C. to approximately 100° C., for a period of approximately 2 minutes to approximately 15 minutes, in particular approximately 5 minutes to approximately 10 minutes.

13. Process according to one of claims 7 to 12, characterised in that it comprises before the steeping stage:
a stage of washing the starting plant product, in particular in water optionally containing a food disinfectant such as sodium hypochlorite, the said washing stage being optionally followed by a stage of rinsing, in particular in water and,
a spin-drying stage for the plant product, in particular by centrifugation.

14. Process according to one of claims 7 to 13, characterised in that it comprises at the end of the steeping stage a spin-drying stage in order to obtain a partially dehydrated plant product presenting:
a mineral salt content of approximately 5% to approximately 10% by weight relative to the total weight of the said plant product, in particular approximately 7% to approximately 9%,
a glucide or polyol content of approximately 2% to approximately 5% by weight relative to the total weight of the said plant product, in particular approximately 3% to approximately 4%,
a water content of approximately 40% to approximately 60% by weight relative to the total weight of the said plant product,
a water activity of approximately 0.7 to approximately 0.9, in particular approximately 0.75 to approximately 0.85.

15. Process according to one of claims 7 to 14, characterised in that the spin-drying of the plant product obtained at the end of the steeping stage is carried out by centrifugation, at a speed of approximately 300 revolutions per minute to approximately 900 revolutions per minute, for approximately 1 minute to approximately 3 minutes.

16. Process according to one of claims 7 to 15, characterised in that the drying of the plant product obtained at the end of the steeping and spin-drying stage is carried out by blowing of hot air at controlled humidity, at a temperature of approximately 40° C. to approximately 70° C., for a period of approximately 5 minutes to approximately 30 minutes.

17. Process according to one of claims 7 to 16, characterised in that it comprises after the drying stage:
a stage of cutting the substantially dehydrated plant product, followed by a sorting stage to separate the leaves, the stems, and the flowers of the aforementioned plant product,
a stage of packaging of the substantially dehydrated plant product, in particular under normal atmosphere or under vacuum, which can be kept either at the temperature of 18° C. to 22° C., or at 4° C., or at −20° C.

18. Process according to one of claims 7 to 17, characterised in that it comprises the following stages:
washing of the starting plant product, in particular in water optionally containing a food disinfectant such as sodium hypochlorite, the said washing stage being optionally followed by a stage of rinsing, in particular in water and,
spin-drying of the plant product, in particular by centrifugation for example at approximately 300 to approximately 500 rpm, for approximately 1 min to approximately 3 min.
steeping of the original plant product in a steeping solution containing:
approximately 55% to approximately 70% of water, by weight relative to the total weight of the steeping solution, in particular approximately 65% to approximately 70%,
approximately 15% to approximately 30% of mineral salt, by weight relative to the total weight of the steeping solution, in particular sodium chloride, in particular approximately 20% to approximately 25%,
approximately 10% to approximately 30% of glucide, in particular chosen from the group constituted by sucrose, fructose, glucose, lactose or maltose or, approximately 10% to approximately 30% of polyol, in particular sorbitol or glycerol, by weight relative to the total weight of the steeping solution, in particular approximately 14% to approximately 25%, and preferably approximately 14.5% to 16.5% of polyol and preferably approximately 19% to approximately 22% for the glucide, and optionally,
approximately 0.1% to approximately 0.3% of a compound containing the magnesium ion, such as magnesium hydroxide, acetate, carbonate or chloride, in particular 0.2% by weight relative to the total weight of the steeping solution,
the said steeping being carried out at a temperature of approximately 70° C. to approximately 120° C., in particular approximately 85° C. to approximately 100° C., for a period of approximately 2 minutes to approximately 15 minutes, in particular approximately 5 minutes to approximately 10 minutes,
spin-drying of the plant product obtained at the end of the steeping stage, in particular by centrifugation, at a speed of approximately 300 revolutions per minute to approximately 900 revolutions per minute, for approximately 1 minute to approximately 3 minutes, in order to obtain a partially dehydrated plant product,
drying of the partially dehydrated plant product obtained at the end of the spin-drying stage, in particular by blowing of hot air at controlled humidity, at a temperature of approximately 40° C. to approximately 70° C., for a period of approximately 5 minutes to approximately 30 minutes,
in order to obtain a substantially dehydrated plant product,
cutting of the substantially dehydrated plant product, followed by a sorting stage to separate the leaves, the stems and the flowers of the aforementioned plant product, which sorting is for example carried out by vibrating screening and on a densimetric column,
packaging of the substantially dehydrated plant product, in particular under normal atmosphere or under vacuum,
which can be kept either at the temperature of 18° C. to 22° C., or at 4° C., or at −20° C.

19. Substantially dehydrated plant product capable of being obtained by implementation of the process according to one of claims 7 to 18.
